(19) United States
(12) Patent Application Publication
(10) Pub. No.: US 2009/0297455 A1
(43) Pub. Date: Dec. 3, 2009

(54) DEVICE FOR AND A METHOD OF ACTIVATING A PHYSIOLOGICALLY EFFECTIVE SUBSTANCE BY ULTRASONIC WAVES, AND A CAPSULE

(75) Inventors: Jan Suijver, Dommelen (NL); Cornelis Reinder Ronda, Aachen (DE)

Correspondence Address: K&L Gates LLP, P.O. BOX 1135, CHICAGO, IL, 60690, US

(73) Assignee: KONINKLIJKE PHILIPS ELECTRONICS N.V., Eindhoven (NL)

(21) Appl. No.: 12/375,940

(22) PCT Filed: Aug. 7, 2007
(86) PCT No.: PCT/IB07/53109

§ 371 Date: Feb. 2, 2009

(30) Foreign Application Priority Data
Aug. 9, 2006 (EP) 06118680.5

(51) Int. Cl. A61K 49/22; A61B 17/20

(52) U.S. Cl. 424/9.5; 604/22

(57) ABSTRACT

A device (100) for activating a physiologically effective substance (101) by ultrasonic waves (103, 105), the device comprising an ultrasonic transducer (102) adapted to generate ultrasonic waves (103), a focusing element (104) adapted to focus the generated ultrasonic waves (103), and an adjustment unit (107) adapted to adjust a position (106) to which the focusing element (105) focuses the generated ultrasonic waves (103) in a manner that the focused ultrasonic waves (103) are bringable in interaction with the physiologically effective substance (101) at the adjusted position (106).

### FIELD OF THE INVENTION

The invention relates to a device for activating a physiologically effective substance by ultrasonic waves.

The invention further relates to a method of activating a physiologically effective substance by ultrasonic waves.

Moreover, the invention relates to a capsule.

### BACKGROUND OF THE INVENTION

Ultrasonic waves may be used as an energy source for generating light which, in turn, may be used for an activation of a chemical reaction.

US 2003/0147812 discloses a system for the targeted initiation or deactivation of chemical reactions by an acoustic energy source in a host. A system for the targeted delivery of drugs, diagnostic agents and other compounds using an acoustic energy source is also disclosed.

However, the accuracy of such a device for activating a physiologically effective substance may still be insufficient under undesired circumstances.

### OBJECT AND SUMMARY OF THE INVENTION

It is an object of the invention to efficiently activate a physiologically effective substance.

In order to achieve the object defined above, a device for activating a physiologically effective substance by ultrasonic waves, methods of activating a physiologically effective substance by ultrasonic waves, and a capsule according to the independent claims are provided.

According to an exemplary embodiment of the invention, a device for activating a physiologically effective substance by ultrasonic waves is provided, the device comprising an ultrasonic transducer adapted to generate ultrasonic waves, a focusing element adapted to focus the generated ultrasonic waves, and an adjustment unit adapted to adjust a position to which the focusing element focuses the generated ultrasonic waves in a manner that the focused ultrasonic waves are bringable in interaction with the physiologically effective substance at the adjusted position.

According to another exemplary embodiment of the invention, a method of activating a physiologically effective substance by ultrasonic waves is provided, the method comprising generating ultrasonic waves, focusing the generated ultrasonic waves, and adjusting a position to which the generated ultrasonic waves are focused in a manner that the focused ultrasonic waves are brought in interaction with the physiologically effective substance at the adjusted position.

According to still another exemplary embodiment of the invention, a capsule is provided comprising an encapsulation and a compartment formed in the encapsulation accommodating a physiologically effective substance, wherein the encapsulation is adapted to be influenced by ultrasonic waves in a manner to expose the physiologically effective substance to an environment, and wherein the physiologically effective substance is adapted to be activated under the influence of the ultrasonic waves.

According to yet another exemplary embodiment of the invention, a method of activating a physiologically effective substance by ultrasonic waves is provided, the method comprising influencing an encapsulation of a capsule by ultrasonic waves in a manner to expose the physiologically effective substance accommodated in a compartment formed in the encapsulation to an environment, and activating the physiologically effective substance under the influence of the ultrasonic waves.

The term “activating” may particularly denote modifying at least one physical, biological, or chemical property of a substance in a manner that the physiologically effective substance is brought in a state to be capable to generate a desired influence on the environment, for instance to destroy specific cells, to initiate specific chemical reactions, etc.

The term “physiologically effective substance” may particularly denote any substance which is capable of having an impact on a (specific part of) human being, an animal, a plant, or bacteria. The physiologically effective substance may be inert or inactive with regard to its specific function in an inactive configuration, but may be reconfigured (for instance by ultrasonic waves) to be brought into an active configuration in which it is capable to fulfill its specific function.

The term “ultrasonic waves” may particularly denote sound having frequencies above normal human hearing, generally accepted to be at 20 kHz to 2 MHz and above, but also extended down to the 5 kHz to 20 kHz range in certain processing applications (like subsonic, supersonic, or transsonic, which have to do with the speed of sound).

The term “focusing” may particularly denote specifically influencing ultrasonic waves to concentrate or spatially limit a beam of ultrasonic waves.

The term “interference” may particularly denote the constructive and/or destructive superposition of two or more wavefronts that may have different phases and/or different frequencies. In an interferometer, the wavefronts may be brought in interference.

The term “sonoluminescence” may particularly denote the emission of short bursts of light from imploding bubbles in a medium (like a liquid) when excited by sound. Sonoluminescence may occur whenever a sound wave of sufficient intensity induces a gaseous cavity within a medium (like a liquid) to quickly collapse. This cavity may take the form of a pre-existing bubble, or may be generated through a process known as cavitation. Sonoluminescence can be made to be stable, so that a single bubble will expand and collapse over and over again in a periodic fashion, emitting a burst of light each time it collapses. For this to occur, a standing acoustic wave may be set up within a medium (like a liquid), and the bubble may sit at a pressure anti-node of the standing wave. The frequencies of resonance may depend on the shape and size of the container in which the bubble is contained.

The term “photodynamic therapy” (PDT) may particularly denote a treatment that combines a light source (and/or an ultrasonic sound source) and a photosensitizing agent (a drug that is activated by light), for instance to destroy cancer. Examples for a photosensitizer (precursor) are aminolevulinic acid (ALA) or methyl aminolevulinate.

According to an exemplary embodiment of the invention, a medical device may be provided in which a location to which ultrasonic waves are to be focused may be defined (for instance in a user-defined manner), and ultrasonic waves emitted by an ultrasonic wave generator may then be focused in accordance with the adjustment of the spatial position. Therefore, a spatially limited or restricted region may be defined in which a sufficiently high acoustic density is present, so that resonance effects or energy deposition or transfer effects may occur, which may serve for activating a physiologically effective substance positioned very close to the selected focusing position.

According to an exemplary embodiment, it may be possible that ultrasonic energy is directly used for exciting the physiologically effective substance from an inactive state into an active state, for instance via an energy transfer scheme which converts ultrasonic energy without intermediate light generation procedures into excitation energy of the physiologically effective substance. This may be made possible by ensuring that the distance between the ultrasonic focus and the substance to be excited is sufficiently small, for instance smaller than 10 nm, preferably smaller than 5 nm. In contrast to conventional approaches, the transfer may be free of any generation or use of electromagnetic radiation (like light pulses), since at sufficiently small distances between an ultrasonic resonance centre and the physiologically effective substance to the activated, a direct transfer of the ultrasonic energy may be possible.

Such a resonance phenomena or, more generally, a constructive interference of two ultrasonic wave contributions, may be made possible with two or more ultrasonic transducers, being operated at slightly different frequency values. Such a difference (difference between two ultrasonic frequencies divided by one of the frequencies or divided by an average value of the two frequencies) may be in the order of magnitude of percents to per mills. An appropriate frequency difference may be dependent on the velocity of sound in the respective medium.

With two identical frequencies, a standing wave may be generated. However, when providing the acoustic frequencies of the two transducers (located at a distance from one another) to be slightly different, a constructive interference may be promoted, thereby promoting focusing effects.

Such a spatial focusing of ultrasonic power may allow to spatially restrict the position of ultrasonic energy concentration (possibly without light generation), so that acoustic energy may be directly transferred to the physiologically effective substance, for instance a photosensitizer. According to exemplary embodiments, the term “photosensitizer” may be related to a conventional use of such substances, wherein embodiments of the invention may use such substances but do not necessarily supply photons for exciting the photosensitizer, but may excite the photosensitizer using acoustic energy.

In order to allow such a direct use of acoustic energy for an excitation of the physiologically effective substance, the distance between the focus of the ultrasonic waves and the position of the physiologically effective substance to be chemically modified should be sufficiently small, particularly smaller than 10 nm, more particularly smaller than 5 nm. Then, the overlap or interaction of the physiologically effective substance and the focused ultrasonic waves is strong enough to allow the direct conversion of the ultrasonic energy into an excitation, without involving an optical luminescence effect. Consequently, the degree of efficiency of the energy transfer may be high according to exemplary embodiments of the invention.

According to an exemplary embodiment, it is possible to provide a transducer system within or without a body of a patient to be treated. For instance, the transducer may be attached to an endoscope or catheter, which may be guided into the body of a patient.

However, in order to allow the concentration or the density of ultrasound to be sufficiently high, an adjustment unit may allow to accurately determine a position at which the focus shall be located.

According to an exemplary embodiment, a photodynamic therapy using focused ultrasound may be provided.

Exemplary embodiments of the invention are related to a method to excite a photosensitizer used in photodynamic therapy, by using sonoluminescence generated through focused ultrasound. This way, efficient excitation of the photosensitizer can be obtained in a non-invasive manner. In addition, this method also allows treatment of inner (organ) tissue, without the need of mechanically damaging the body. Further, such a method may have, if at all, fewer side effects than treatments based on medication.

Many people are interested in photodynamic therapy as a patient specific treatment. In conventional photodynamic therapy, either a photosensitizer or a metabolic precursor may be administered to the patient. The tissue to be treated may be exposed to light suitable for exciting the photosensitizer. When the photosensitizer and an oxygen molecule are in close proximity, energy transfer can take place that allows the photosensitizer to relax to its ground singlet state, and which creates an excited singlet state oxygen molecule. Singlet oxygen is a very aggressive chemical species and may rapidly react with any nearby biomolecule. The specific targets depend heavily on the photosensitizer chosen. Ultimatively, these destructive reactions may result in cell killing through apoptosis. Some photosensitizers, like ALA, absorb specifically in rapidly dividing tissue (like cancerous tissue), resulting in a beneficial (spatial) specificity.

However, according to such a conventional approach, a problem may occur that the human body is not transparent for the majority of all optical frequencies which are needed for this treatment. In fact, the body has an optical window in the 800 nm to 1200 nm range only, and only a radiation of this wavelength can penetrate several centimetres in the body. As a result, it is very difficult to efficiently excite the photosensitizer usefully when the tissue to treat is located in the body of the patient. This fact may conventionally limit the applicability of photodynamic therapy as a treatment.

Having the above considerations in mind, exemplary embodiments of the invention may use focused ultrasound inside the body. When suitable wavelengths and intensities are chosen, the phenomenon of sonoluminescence can occur. Sonoluminescence may result in intense visible light at exactly the desired location, thus greatly improving the excitation efficiency for the photosensitizer.

The focusing of ultrasonic radiation can be achieved using an array of small transducers (for instance with a suitable phase difference), liquid crystal lenses, or using a fluid lens that is able to focus ultrasound (see WO 2005/122139 A2).

The ultrasound source can be either outside the patient, or inside, when placed on the tip of an endoscope. In the focal point of the ultrasound, the intensity (and therefore the pressure) may become high enough to induce sonoluminescence. The effects of sonoluminescence are very well established: During the implosion of a microscopic bubble within the solution, a flash of light of duration of 10-100 ps is emitted. The exact wavelength and bandwidth of the light depend on the physical characteristics of the liquid as well as on the gases dissolved in the liquid. By matching the absorption spectrum of the photosensitizer with the emission spectrum of the sonoluminescence, efficient excitation of the photosensitizer can be obtained. This translates to strong energy transfer to the treatment side chosen for the photodynamic therapy.

Therefore, according to an exemplary embodiment, sonoluminescence may be used in (non-invasive) photodynamic therapy. However, other exemplary embodiments may substitute such a sonoluminescence by a direct transfer of acoustic energy to the physiologically effective substance to be excited, without the generation of electromagnetic radiation like light.

However, exemplary embodiments may use ultrasound to generate sonoluminescence. Focusing elements may be used to generate focused ultrasound. It is possible to use phased transducer arrays and/or liquid crystal lenses and/or fluid lenses to generate focused ultrasound to excite a physiologically effective substance. Furthermore, an endoscope may be equipped with such a focused ultrasound generating unit.

Utilization of energy transfer instead of the generation of light followed by optical absorption by the photodynamic agents may be a characteristic of an exemplary embodiment of the invention. Energy transfer is used in fluorescence lighting and can be very efficient (almost 100%). In such a case, the energy generated by the sonoluminescence process may be transferred immediately to a photodynamic agent. To this end, the distance between the location where the light is generated and the photodynamic agent has to be short (in the order of 10 nm). To realize this, the photodynamic agent may be administered in spheres, beads, pellets, etc., or in other capsules in which the sonoluminescence is generated and which are destroyed as a consequence of coupling to the acoustic waves, to enable the excited photodynamic molecules to reach the ill tissue. This may also increase the number of appropriate (photodynamic) materials, as the requirements on the optical absorption strength can be less stringent. Alternatively, this relaxes any inconvenience for patients with respect to exposure to daylight. This is desirable from the point of view of the patient as a patient subjected to a photodynamic treatment generally needs to avoid daylight for an extended period. This also reduces societal costs for such a treatment (for instance because the persons involved can go back to work earlier).

It is also possible to use beads, etc., in which both the photodynamic agent and oxygen are accommodated. In such cases, singlet oxygen can be generated within the bead. Moreover, such a scheme may even generate singlet oxygen without a photodynamic compound (also based on energy transfer), enabling a completely new therapeutic system with, if at all, less side effects (as no photodynamic agents need to be used). This is desirable from the point of view of the patient as a patient subjected to a photodynamic treatment generally needs to avoid daylight for an extended period. This also reduces societal costs for such a treatment (for instance because the persons involved can go back to work earlier).

By using two transducers with the same frequency (of generated mechanical waves), a standing wave can be realized. By using transducers (two or more) with a slightly different frequency, the sound wave can be concentrated to a large extent in a very small region. In this way, damage to healthy tissue can be reduced or even minimized. In addition, the energy input at the desired locations can be increased, also because healthy tissue is involved less.

It is also possible to add photodynamic therapy features to a catheter adapted for insertion into the body.

Therefore, it is possible to encapsulate the agent used for activating the physiologically effective substance, for instance using a resonance phenomena. When a large amount of ultrasonic energy impinges on the encapsulation, the agent contained therein may be exposed to the environment, by eliminating, destroying or otherwise removing the encapsulation. This may be particularly advantageous in combination with a concentration or focusing of the ultrasonic wave.

Next, further exemplary embodiments of the device will be explained. However, these embodiments also apply for the methods and for the capsule.

The focusing element may comprise at least one additional ultrasonic transducer adapted to generate ultrasonic waves and adapted to be operated in combination with the ultrasonic transducer in a manner to bring ultrasonic waves generated by the ultrasonic transducer and ultrasonic waves generated by the at least one additional ultrasonic transducer in constructive interference at the position in which the ultrasonic waves are focused. Therefore, superposing ultrasonic waves generated by two spatially separated ultrasonic transducers may allow to make use of constructive interference or even resonance phenomena, thereby increasing the focused supersonic energy per volume unit. It is possible to use, altogether, 2, 3, 4, 5, or even more ultrasonic transducers, thereby allowing to refine the spatial distribution of the ultrasonic energy.

The ultrasonic transducer and the at least one further ultrasonic transducer may be adapted to generate ultrasonic waves which are frequency-shifted and/or phase-shifted with respect to one another. By adjusting the plurality of ultrasonic transducers to emit ultrasonic waves at slightly different frequencies, it may be possible to selectively steer or control superposition or resonance phenomena. Additionally or alternatively, a small phase-shift between the supersonic waves emitted by the two or more transducer elements may be generated, thereby having a further parameter for adjusting the superposition properties.

The frequency-shift may be in the range between essentially 0.1% and essentially 10%, particularly in the range between essentially 0.5% and essentially 2%, more particularly in the range between essentially 1% and essentially 5%. These parameters may be adjusted or even tuned (by a human operator or automatically) to obtain a proper result. Therefore, the absolute value of the frequency and/or phase-shift may vary over a broad range, but may be very small in comparison to the absolute values of frequency and/or amplitude of the ultrasonic waves.

The ultrasonic transducer and the at least one further ultrasonic transducer may be adapted to be movable (for instance shiftable and/or tiltable) with respect to one another. Therefore, by adjusting the geometry parameters of the ultrasound generation system, that is to say the distance between the transducers and/or the angular relationship between the transducers, the superposition scheme may be further refined. It is also possible that the amplitude of the emitted ultrasound waves is matched to desired conditions. Also this measure may allow to influence the interference properties.

The focusing unit may comprise an ultrasonic device adapted to variably refract the generated ultrasonic waves to the adjusted position. Such a device may be operated in combination with one or a plurality of transducers. An ultrasonic lens may be denoted as an arrangement of different media separated by a border line, which different media have different ultrasonic sound propagating velocities. This may allow to construct an ultrasonic lens, similarly as in the case of optical lenses, capable of redirecting and/or focusing ultrasonic waves. For example, such an ultrasonic lens may be a liquid crystal lens, that is to say a lens using liquid crystal materials, or may be a fluid focus lens (comprising a displaceable curved boundary between two fluid media having different ultrasonic propagation velocities). The term “fluid focus” lens may particularly denote an acoustic device with variable refraction properties (i.e.: variable focal length and/or variable deflection properties), as disclosed in WO 2005/122139 A2. Specifically with regard to the description of fluid focal lenses, the disclosure of this document is incorporated by reference into the disclosure of this patent application.

The device may further comprise an endoscope on/in which at least one of the group consisting of the ultrasonic transducer, the adjustment unit and the focusing element may be mounted. For example, such an endoscope may be inserted into a body of a patient via a catheter. The catheter may be inserted as a hollow tube in a body lumen, and the endoscope may then be guided through the catheter to a position of interest within the lumen. By taking this measure, the distance between the ultrasonic sound generation, focusing and position adjustment on the one hand and the tissue to be treated on the other hand may be reduced, allowing a further refined adjustment of the processes. However, as an alternative to such an invasive procedure, a non-invasive procedure may also be performed in which a part or all of the components of the device are located outside of a patient's body.

In the following, further exemplary embodiments of the method will be explained. However, these embodiments also apply for the device and for the capsule.

The method may comprise focusing the generated ultrasonic waves to the adjusted position to thereby activate the physiologically effective substance by sonoluminescence. The term “sonoluminescence” may denote the emission of light pulses from imploding bubbles in a liquid when excited by sound. Therefore, the physiologically effective substance may be excited via such an electromagnetic radiation.

However, as an alternative to this embodiment, it is possible that the generated ultrasonic waves are focused to the adjusted position to thereby activate the physiologically effective substance by a direct interaction between the focused ultrasonic waves and the physiologically effective substance without involving electromagnetic radiation. In such a scenario, the distance between an ultrasonic focus on the one hand and the physiologically effective substance on the other hand should be sufficiently small, particularly smaller or equal than 5 nm, so that the deposition of ultrasonic energy directly promotes the excitation of the physiologically effective substance, without meanwhile generating electromagnetic radiation. Therefore, the energy transfer may be much more efficient.

The method may comprise activating a photosensitizer as the physiologically effective substance by ultrasonic waves. Such a photosensitizer may conventionally be excited by photons, that is to say by electromagnetic radiation. However, it is also possible, according to exemplary embodiments of the invention, that such a photosensitizer is directly excited or activated using the mechanical energy of the ultrasonic waves.

In the following, exemplary embodiments of the capsule will be explained. However, these embodiments also apply to the device and to the methods.

The physiologically effective substance may be adapted to be activated to expose a radical, particularly an oxygen radical, under the influence of the ultrasonic waves. Therefore, when the physiologically effective substance is excited by the ultrasonic energy directly or indirectly via light pulses generated due to the sonoluminescence, this energy may be used to ionize a surrounding material, like oxygen, to thereby generate a radical. Such a radical is chemically very aggressive and may destroy surrounding tissue, particularly may selectively destroy specific tissue, like cancer tissue.

However, according to another exemplary embodiment, the capsule may comprise a further compartment formed in the encapsulation accommodating a further substance, wherein the physiologically effective substance is adapted to be activated under the influence of the ultrasonic waves when being brought in contact with the further substance. In other words, the energy of the ultrasonic waves may be used directly or indirectly (via the generation of electromagnetic radiation) and intentionally to destroy the encapsulation, thereby bringing the physiologically effective substance and the further substance in functional contact to one another. A chemical reaction or an energy transfer may occur, so that the two components generate substances or radiation which harms surrounding tissue, thereby selectively destroying tissue in the environment.

The compartment and the further compartment may be separated from one another. In other words, the capsule may have two or more compartments separated by a wall or the like, wherein the wall may be destroyed under the influence of a significantly strong acoustic wave, thereby promoting functional contact of the two components which are accommodated in the two compartments.

For instance, the physiologically effective substance may be a photosensitizer, and the further substance may be oxygen. A mixture of these components under the influence of a sufficient amount of energy may allow for a photodynamic therapy.

The aspects defined above and further aspects of the invention are apparent from the examples of embodiment to be described hereinafter and are explained with reference to these examples of embodiment.

### BRIEF DESCRIPTION OF THE DRAWINGS

The invention will be described in more detail hereinafter with reference to examples of embodiment but to which the invention is not limited.

FIG. 1, FIG. 2, FIG. 6, FIG. 7 illustrate devices according to exemplary embodiments of the invention.

FIG. 3 to FIG. 5 illustrate capsules according to exemplary embodiments of the invention.

### DESCRIPTION OF EMBODIMENTS

The illustration in the drawing is schematically. In different drawings, similar or identical elements are provided with the same reference signs.

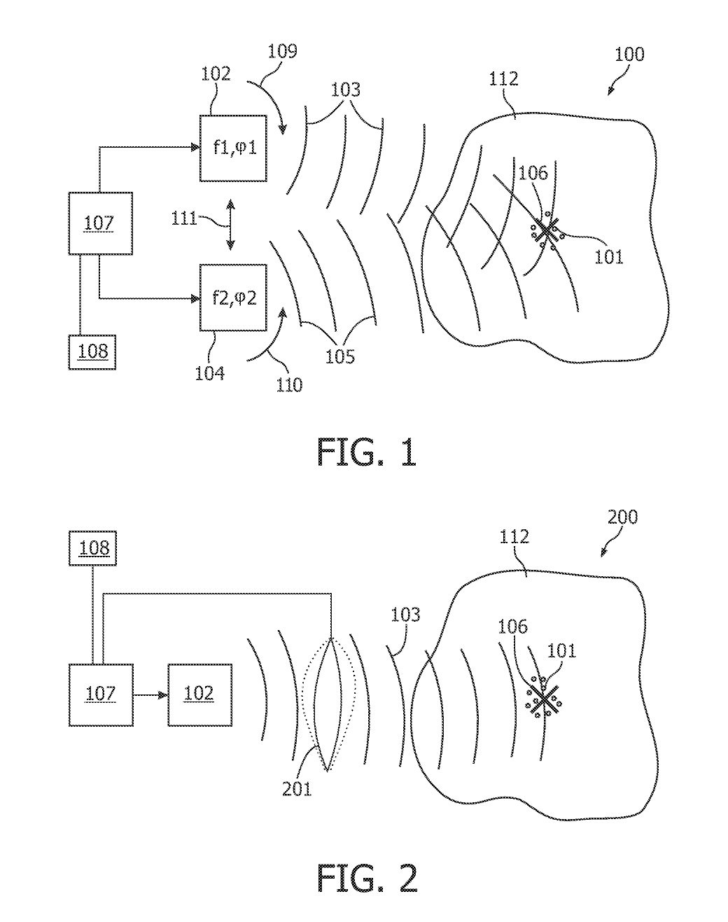

In the following, referring to FIG. 1, a device 100 for activating a physiologically active substance 101 which has previously been administered to a patient (schematically indicated with reference numeral 112). The activation occurs by ultrasonic waves, as will be described in the following in more detail.

The device 100 comprises a first ultrasonic transducer 102 adapted to generate ultrasonic waves 103. The ultrasonic waves 103 are described by a first frequency f₁, and by a first phase characteristic φ₁. Furthermore, a second ultrasonic transducer 104 is foreseen which is also adapted to generate ultrasonic waves 105. The ultrasonic waves 105 are described by a second frequency f₂, and by a second phase characteristic φ₂.

As can be taken from FIG. 1, the transducers 102, 104 are arranged (with regard to distance and emission angle) so that the ultrasonic waves 103, 105 can be brought in interference to one another, and particularly in such a manner that they are focused to a predetermined position 106 which is as close as possible to the physiologically effective substance 101 to be activated. The physiologically effective substance 101 may have been administered specifically to a selected portion (for instance a cancerous organ) of the patient 112. Therefore, the combination of the first and second transducer 102, 104 serves as a focusing unit adapted to focus the generated ultrasonic waves 103, 105 to an adjustable position 106.

A central processing unit (CPU) 107 is provided as a central control unit or adjustment unit and allows, on the basis of instructions which may be provided by an algorithm or by a human operator, to adjust a position 106 to which the focusing element 102, 104 focuses the generated ultrasonic waves 103, 105 in a manner that the focused ultrasonic waves are bringable in interaction with the physiologically effective substance 101 at the adjusted position 106.

Furthermore, a user input/output device 108 is shown via which a user may input operational parameters or conditions or instructions. The input/output device 108 may comprise a display unit like a liquid crystal display, a plasma display or a cathode ray tube. Furthermore, input elements (not shown) may be provided in the user input/output device 108, like a joystick, a keypad, a track ball or even a microphone of a voice recognition system.

By operating the input/output device 108, it is possible for a human operator to define an operation mode of the control unit 107, particularly to define a position 106 and/or frequencies f₁, f₂, φ₁, φ₂ used by the transducers 102, 104 to generate the ultrasonic waves 103, 105.

In other words, the focusing mechanism 102, 104 comprises the transducer element 102 and the further transducer element 104 which is also adapted to generate ultrasonic waves 105 (having a frequency f₂, and a phase property denoted with φ₂) and adapted to be operated in combination with the ultrasonic transducer 102 in a manner to bring the ultrasonic waves 103, 105 in constructive interference at the position 106 at which the ultrasonic waves 103, 105 are focused. For this purpose, the ultrasonic waves 103, 105 may be frequency-shifted to one another, so that |f₁−f₂| may be in the order of magnitude of several per mills.

As indicated schematically with arrows 109, 110, the transducer devices 102, 104 may be tilted with respect to one another, to provide a further adjustment parameter to adjust the superposition properties.

Furthermore, as indicated schematically by an arrow 111, the distance between the transducer elements 102, 104 may be modified in 1, 2, or 3 direction(s) of the Cartesian coordinate system, so as to adjust the geometrical arrangement of the transducers 102, 104 to match desired superposition properties.

At the position 106, the ultrasonic waves 103, 105 are constructively interfering so as to deposit a significant amount of acoustic energy in a spatially restricted portion around the position 106. This activates the physiologically active substance 101, which may be brought in an activation state by sonoluminescence or by a direct conversion of the acoustic energy into excitation energy without involving electromagnetic radiation.

FIG. 2 shows another embodiment of a device 200 for activating a physiologically effective substance 101.

In the embodiment of FIG. 2, only a single ultrasonic transducer 102 is shown which is again controlled by a control unit 107. The ultrasonic waves 103 which are emitted by the transducer 102 may be adjusted with respect to frequency, amplitude and/or phase properties.

In addition to that, an ultrasonic lens 201 (for instance in the manner as disclosed by WO 2005/122139 A2) is foreseen to focus the ultrasonic waves 103 to a specific focal point or to the predetermined position 106. For this purpose, a control signal may be supplied to the lens 201 (having variable focusing properties) by the control unit 107.

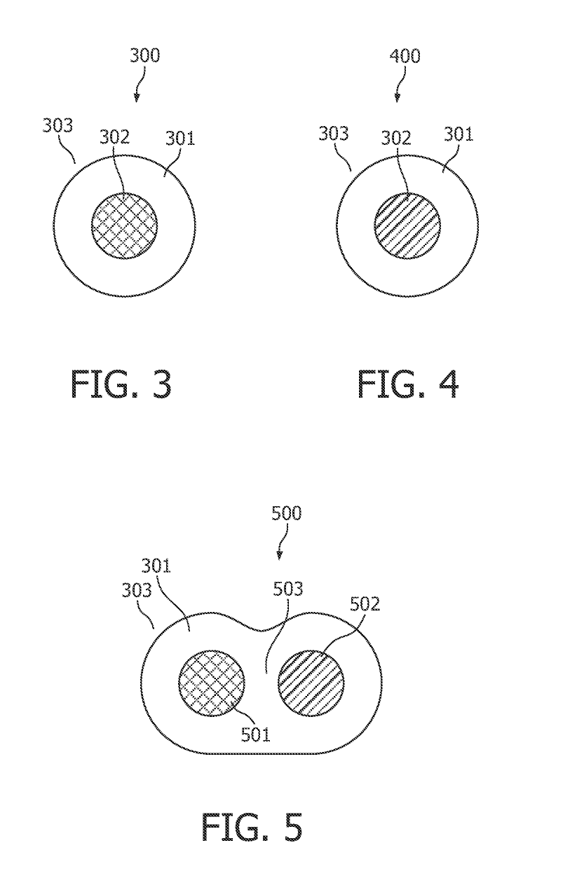

FIG. 3 shows a capsule 300 according to an exemplary embodiment of the invention.

The capsule 300 comprises an encapsulation 301 which may be made of a polymer material or the like. In an interior of the encapsulation 301, a compartment 302 is formed which accommodates a physiologically effective substance, in the present embodiment pre-formed oxygen radicals. When ultrasonic waves 103 and/or 105 of a sufficient amplitude or intensity are irradiated onto the capsule 300, the encapsulation 301 will be intentionally destroyed by these ultrasonic waves 103 and/or 105, thereby exposing the physiologically active substance, namely the oxygen radicals, to an environment 303.

By taking these measures, the physiologically effective substance may be separated in an encapsulated operation state from surrounding tissue, and only when acoustic energy of sufficient power or intensity is irradiated onto the capsule 300, the encapsulation 301 is destroyed and the physiologically effective substance is activated so as to selectively and intentionally destroy surrounding tissue (for instance ill tissue, like cancerous tissue).

FIG. 4 shows another embodiment of a capsule 400.

The capsule 400 differs from the capsule 300 in that in the embodiment of FIG. 4 a photosensitizer material is provided in the compartment 302. The photosensitizer is in an inactive state and may be excited only when ultrasonic sound 103 and/or 105 impinges onto the encapsulation 301, destroying the encapsulation 301 and providing also the physiologically effective substance, namely the photosensitizer, with sufficient energy to be excited. The excited photosensitizer may then react with oxygen in an environment to ionize the oxygen, so that the oxygen in the environment is converted into activated aggressive oxygen radicals.

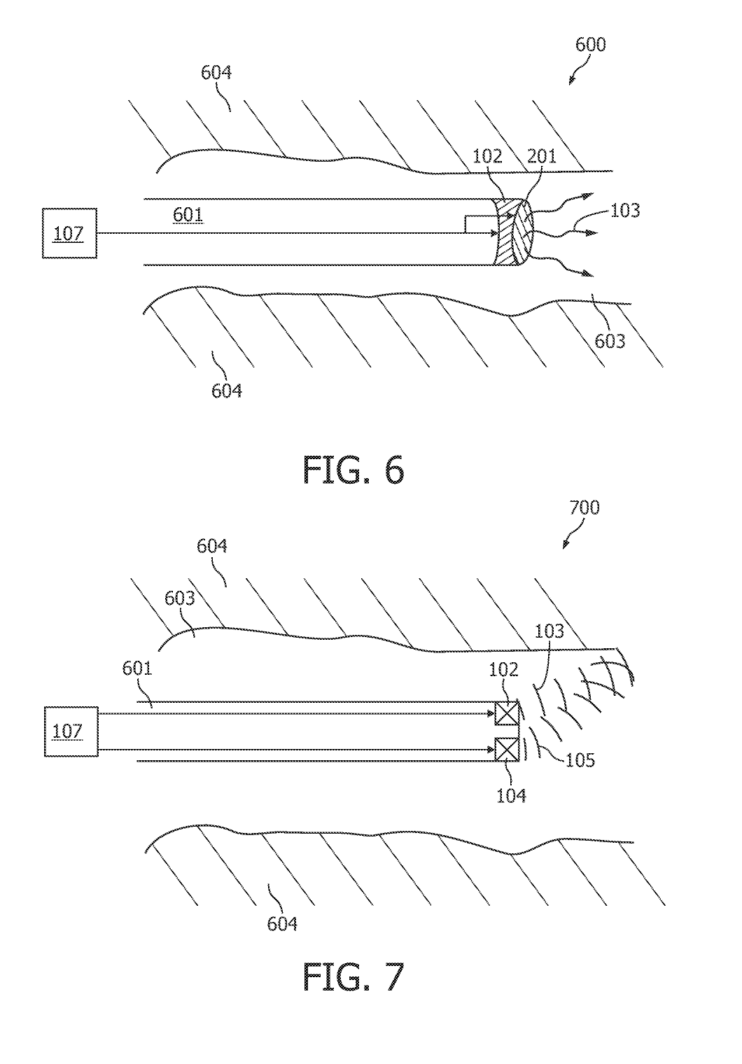

FIG. 5 shows a capsule 500 according to another exemplary embodiment of the invention.

In the case of the capsule 500, the encapsulation 301 forms a first compartment 501 and a second compartment 502, wherein the first compartment 501 and the second compartment 502 are separated by a wall-like element 503.

Upon irradiation of the capsule 500, the encapsulation 301 is destroyed and the oxygen molecules provided in the first compartment 501 and the photosensitizer provided in the second compartment 502 are brought in functional contact to one another, so that a pre-defined chemical reaction may occur which may generate a substance in the surrounding 303 which may treat the surrounding in a desired manner.

FIG. 6 shows another embodiment of a device 600 for activating a physiologically effective substance by ultrasonic waves.

In the embodiment of FIG. 6, an endoscope 601 (a catheter may be used as well) is shown which is inserted into a body lumen 603, and is therefore surrounded by tissue 604 of a human being.

The embodiment of FIG. 6 is similarly to the embodiment of FIG. 2. However, the components 102, 201 are mounted on a tip of the endoscope 601.

FIG. 7 shows a device 700 according to a further exemplary embodiment of the invention.

FIG. 7 is similar to the embodiment of FIG. 1, namely provides two transducers 102, 104 which are, however, mounted on a tip of an endoscope 601 located in a body lumen 603, that is to say are surrounded by tissue 604. The control of the transducers 102, 104 occurs from the remotely located central control unit 107. In other words, the central control unit 107 is located outside of the body, but may alternatively be located inside of the body as well.

It should be noted that the term “comprising” does not exclude other elements or features and the “a” or “an” does not exclude a plurality. Also elements described in association with different embodiments may be combined.

It should also be noted that reference signs in the claims shall not be construed as limiting the scope of the claims.

1.-8. (canceled)

9. A method of activating a photodynamic agent by ultrasonic waves, the method comprising
generating the ultrasonic waves;
focusing the generated ultrasonic waves to produce sonoluminescence by breaking a bubble in the vicinity of a photodynamic agent; and
activating the photodynamic agent by sonoluminescence.

10. The method of claim 9,
comprising focusing the generated ultrasonic waves to the position of the photodynamic agent to thereby activate the photodynamic agent by sonoluminescence.

11. The method of claim 9,
comprising focusing the generated ultrasonic waves to the position of the photodynamic agent to thereby activate the photodynamic agent by a direct interaction between the photons of the sonoluminescence and the photodynamic agent without involving electromagnetic radiation.

12. The method of claim 9,
comprising focusing the generated ultrasonic waves to the position of the photodynamic agent to thereby activate the photodynamic agent by a direct ultrasonic energy transfer from the focused ultrasonic waves to the photodynamic agent.

13. The method of claim 9,
comprising activating a photosensitizer as the photodynamic agent activated by ultrasonic waves.

14. A capsule, comprising
an encapsulation;
a compartment formed in the encapsulation accommodating a photo-activated physiologically effective substance;
wherein the encapsulation is adapted to be influenced by ultrasonic waves in a manner to expose the photo-activated physiologically effective substance to photon environment;
wherein the photo-activated physiologically effective substance is adapted to be activated under the influence of the ultrasonic waves.

15. The capsule of claim 14,
wherein the photo-activated physiologically effective substance is adapted to be activated to generate or expose a radical, particularly an oxygen radical, under the influence of the ultrasonic waves.

16. The capsule of claim 14,
comprising a further compartment formed in the encapsulation accommodating a further substance;
wherein the photo-activated physiologically effective substance is adapted to be activated under the influence of the ultrasonic waves when being brought in contact with the further substance.

17. The capsule of claim 16,
wherein the compartment and the further compartment are separated from one another by a wall of the encapsulation.

18. The capsule of claim 16,
wherein the photo-activated physiologically effective substance comprises a photosensitizer, and the further substance comprises oxygen.

19. A method of activating a physiologically effective substance by ultrasonic waves, the method comprising
influencing an encapsulation of a capsule by ultrasonic waves in a manner to expose the physiologically effective substance accommodated in a compartment formed in the encapsulation to an environment of focused ultrasonic waves; and
activating the physiologically effective substance under the influence of sonoluminescence produced by the focused ultrasonic waves.

20. The method of claim 19,
comprising influencing the encapsulation and activating the physiologically effective substance by a direct interaction with the sonoluminescence produced by the focused ultrasonic waves without involving electromagnetic radiation.
